(19) United States
(12) Patent Application Publication
(10) Pub. No.: US 2013/0331832 A1
(43) Pub. Date: Dec. 12, 2013

(54) ELECTRODE CATHETER DEVICE

(71) Applicant: Peter Osypka, Grenzach-Wyhlen (DE)

(72) Inventor: Peter Osypka, Grenzach-Wyhlen (DE)

(21) Appl. No.: 13/908,834

(22) Filed: Jun. 3, 2013

(30) Foreign Application Priority Data
Jun. 6, 2012 (EP) 12004329

(51) Int. Cl. A61B 18/18; A61B 18/14; A61B 18/16

(52) U.S. Cl. CPC A61B 18/18; A61B 18/16; A61B 18/1492
USPC 606/33

(57) ABSTRACT

An implantable electrode catheter device comprising an inner electrode catheter and an outer electrode catheter. The outer electrode catheter including a catheter shaft having at least one electrode at a distal end and a lumen to receive the inner electrode catheter therein. The outer electrode is adjustable or movable relative to the inner electrode catheter in an axial direction. The inner electrode catheter has a fixation element disposed at a distal end. The inner electrode catheter together with the fixation element forms an indifferent electrode whereby radio frequency catheter ablation occurs between the electrode and the indifferent electrode.

### CROSS-REFERENCE TO RELATED APPLICATIONS

This application claims under 35 U.S.C. §119(a) priority to and the benefit of European Patent Application No. EP12004329 filed Jun. 6, 2012, which is incorporated by reference herein in its entirety.

### BACKGROUND OF THE INVENTION

1. Field of the Invention

The present invention relates to an ablation device and more particularly for the ablation of tissue in a body cavity of a human or an animal, especially used to treat atrial fibrillation.

2. Description of Related Art

Atrial fibrillation is a frequent finding especially in elderly patients and occurs when the normal electrical impulses that are generated by the SA node are overwhelmed by disorganized electrical impulses in the atria. These disorganized impulses cause the muscles of the upper chamber of the heart to fibrillate and this leads to the conduction of irregular impulses to the ventricles and may lead to acute hemodynamic instability.

In the treatment of atrial fibrillation, for example, a radio frequency (RF) ablation catheter equipped with a number of electrodes can be brought into contact with cardiac tissue according to the so called “Maze procedure” for creating one or more ablation points or an ablation path along the tissue especially around the pulmonary veins. To create an ablation path the catheter must have a large number of electrodes and can thus only be produced at high costs. Furthermore, the exact guidance of the catheter is difficult and time consuming. As the guidance is done under x-ray control the exposure to x-rays is high.

U.S. Patent Application Publication No. 20090312755 discloses an ablation device comprising an elongate shaft and a positioning mechanism and an energy delivery element adjacent the distal end of the shaft said energy delivery element is adapted to create a zone of ablation.

Such conventional methods and systems have generally been considered satisfactory for their intended purpose. However, there is still a need in the art for an electrode catheter device that allows for improved treatment of atrial fibrillation by creating an ablation path that allows access to the various locations in the heart. The present invention provides a solution for these problems.

### SUMMARY OF THE INVENTION

The subject invention is directed to a new and useful electrode catheter device.

The present embodiment provides an implantable electrode catheter device comprising an inner electrode catheter and an outer electrode catheter. The outer electrode catheter includes a catheter shaft having at least one electrode at a distal end and a lumen to receive the inner electrode catheter therein. The outer electrode is adjustable or movable relative to the inner electrode catheter in an axial direction. The inner electrode catheter has a fixation element disposed at a distal end. The inner electrode catheter together with the fixation element forms an indifferent electrode whereby radio frequency catheter ablation occurs between the electrode and the indifferent electrode. A high frequency voltage is applied between the electrode and the indifferent electrode thereby heating the subjacent tissue and creating an ablation path.

The fixation element is preferably a helical screw or a magnet and allows the fixation of the electrode at any place in the left atrium or if desired in the right atrium or the ventricles.

The movable outer catheter carries at least one electrode for energy absorption or energy release. The electrode is made of metal or of conductive plastic and is located at the wall of the left atrium or if desired the wall of the right atrium. The electrode of the outer electrode catheter may be designed in cylindrical shape or is in shape of an electrically conducting segment.

The ablation temperature may be controlled by a temperature sensor embedded proximate the distal end on the catheter shaft.

The shaft of the outer electrode catheter may include a red light emitting diode (LED) at a distal end thus helping to control the position of the electrode catheter device during the ablation process.

The shaft of the outer electrode catheter may be made of flexible plastic.

In an alternate embodiment, a method for creating an ablation path is disclosed. The method comprising guiding an inner electrode catheter to a desired target in the left atrium of a patient. The inner electrode catheter having an electrically conducting helix. Next, fixing a distal end of the inner electrode catheter at an outer wall of the left atrium with a helical screw such that the electrically conducting helix of the inner electrode catheter and the helical screw from an indifferent electrode. An outer electrode catheter is guided over the inner electrode catheter such that the outer electrode catheter concentrically encases the inner electrode catheter. A shaft of the outer electrode catheter includes an electrode. A high frequency voltage is applied between the electrode of the outer electrode catheter and the indifferent electrode thereby heating subjacent tissue and creating an ablation path.

These and other features of the systems and methods of the subject invention will become more readily apparent to those skilled in the art from the following detailed description of the preferred embodiments taken in conjunction with the drawings.

### BRIEF DESCRIPTION OF THE DRAWINGS

So that those skilled in the art to which the subject invention appertains will readily understand how to make and use the devices and methods of the subject invention without undue experimentation, preferred embodiments thereof will be described in detail herein below with reference to certain figures, wherein:

FIG. 1 is a side view of the electrode catheter device according to the invention implanted in the left atrium of the heart;

FIGS. 2a-2c are detailed views of the catheter device of FIG. 1, showing the inner electrode catheter disposed within the outer electrode catheter;

FIG. 3 is a detailed view of the electrode catheter device with helical screw as shown in FIG. 2a;

FIG. 4 shows the positioning of the inventive electrode catheter device at the outer wall of the left atrium when retracting the catheter during radio frequency ablation; and

FIG. 5 shows the positioning of the inventive electrode catheter device at the outer wall of the left atrium when retracting and rotating the catheter during radio frequency ablation.

### DETAILED DESCRIPTION OF THE PREFERRED EMBODIMENTS

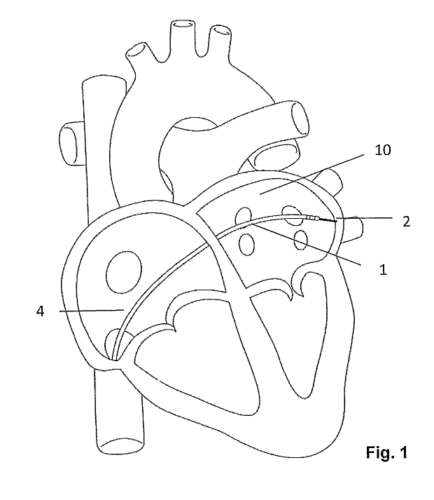

Reference will now be made to the drawings wherein like reference numerals identify similar structural features or aspects of the subject invention. For purposes of explanation and illustration, and not limitation, a partial view of an exemplary embodiment of the electrode catheter device in accordance with the invention is shown in FIG. 1 and is designated generally by reference character 1. The system of the invention can be used for the treatment of atrial fibrillation.

FIG. 1 shows an electrode catheter device 1 implanted in the left atrium 10 of the heart, which is anchored with a helical screw 2 at the wall of the left atrium. The location of the anchor may be at anywhere at the tissue of the atrium. The access to the left atrium 10 may be achieved by transseptal puncture from the right atrium through the intra-atrial septum using known methods.

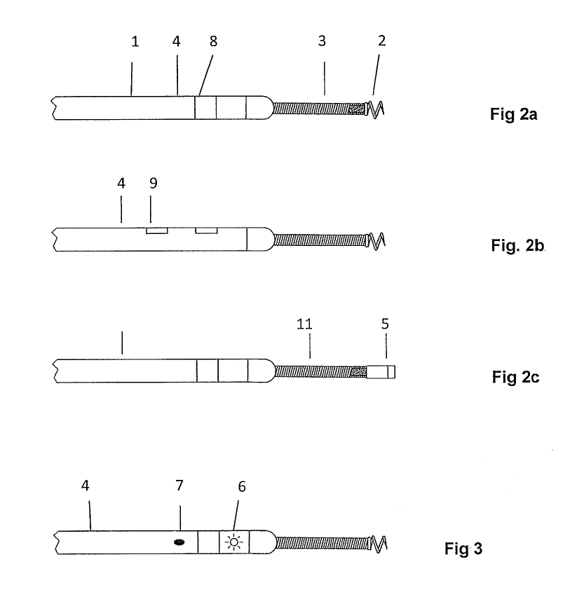

At first the inner electrode catheter 3 is guided to the desired target in the left atrium 10 by means of a steerable catheter device (not shown). When a distal end of the inner electrode catheter 3 has reached its target in the left atrium 10, the inner electrode catheter 3 is fixed at the outer wall of the left atrium 10 by means of a fixation device such as for example by means of a helical screw 2 (FIGS. 2a and 2b) or a magnet 5 (FIG. 2c). and the steerable catheter is retracted. Then the outer electrode catheter 4 is guided over the inner electrode catheter 3 so that the outer electrode catheter 4 concentrically encases the inner electrode catheter 3.

FIG. 2 shows a detailed view of the inventive catheter devices 1 showing the inner electrode catheter 3 disposed within the outer electrode catheter 4. The shaft of the outer electrode catheter 4 carries the electrode 8 which is made from metal or from conductive plastic such as polyurethane or silicone. The shape of the outer electrode is variable. The electrode 8 of the outer electrode catheter 4 may be designed in cylindrical shape (FIGS. 2a, 2c) or is in shape of an electrically conducting segment 9 (FIG. 2b).

The inner electrode catheter 3 preferably comprises an electrically conducting helix shaped electrode 11 or an electrically conducting strand which is torsional rigid.

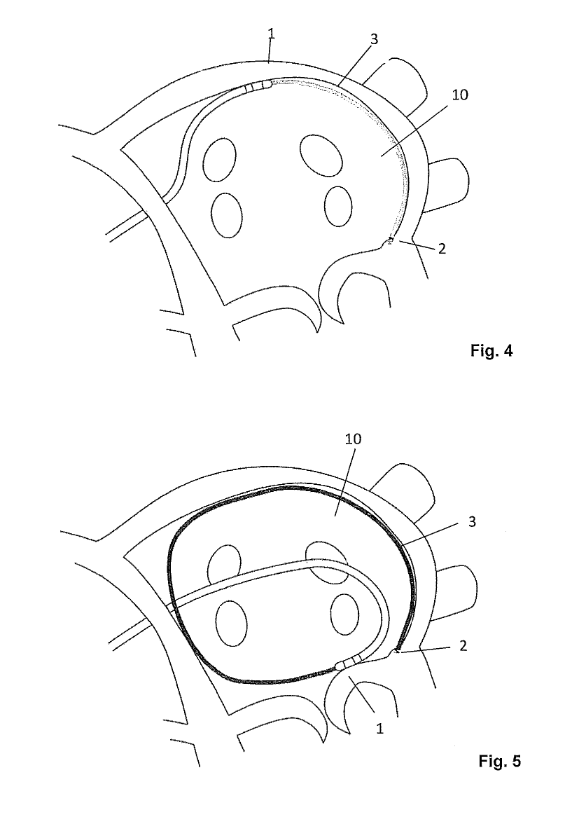

FIG. 3 shows a detailed view of the electrode catheter device 1 with the helical screw 2. The shaft of the outer electrode catheter 4 also includes a red light emitting diode (LED) 6 and/or a temperature sensor 7 embedded proximate the distal end. Thus, the ablation temperature is controlled by receiving feedback from the temperature sensor.

The red light emitting diode 6 shows the position of the electrode catheter device 1 without submitting x-rays to the patient as done in common ablation processes due to an x-ray sensor placed e.g. in the esophagus or in a vein.

FIG. 4 shows the positioning of the inventive electrode catheter device 1 at the outer wall of the left atrium 10 when retracting the catheter during radio frequency ablation. The electrically conductive helix 11 of the inner electrode catheter 3 together with the helical screw 2 forms the indifferent electrode. Between the electrode 8 and the indifferent electrode high frequency voltage of about 500 kHz is applied thus heating the subjacent tissue and thus creating an ablation path. When retracting the outer electrode catheter 4 the surface of the indifferent electrode increases.

It is important that the inner electrode catheter 3 is at any place in the left atrium 10. Depending on the position of the fixation point in the tissue of the left atrium, preferably in the wall it is possible to draw the ablation path in the whole atrium. Each and every part of the atrium can be reached. The possibility of reaching each and every part of the left atrium is further supported by being able to rotate the electrode catheter device 1 when retracting the device as shown in FIG. 5.

FIG. 5 shows the positioning of the inventive electrode catheter device 1 at the outer wall of the left atrium 10 when retracting the catheter during radio frequency ablation. By rotating the outer catheter 4 180 degrees a change of position occurs as shown in FIG. 5. Depending on the position of fixation and on the position where the rotation occurs each part of the left atrium 10 can be reached to create an ablation path. Even the ablation around the pulmonary veins is possible.

If desired, the present ablation process can be combined with a chemical ablation process using sodium chloride. The device described hereinabove is referenced treating heart tissue, however it is understood that the ablation process is not limited to the ablation of heart tissue. Further tissue such as for example kidney tissue or stomach tissue may also be ablated.

The methods and systems of the present invention, as described above and shown in the drawings, provide for an electrode catheter device with superior properties. While the apparatus and methods of the subject invention have been shown and described with reference to preferred embodiments, those skilled in the art will readily appreciate that changes and/or modifications may be made thereto without departing from the spirit and scope of the subject invention.

1. An implantable electrode catheter device comprising:
an inner electrode catheter; and
an outer electrode catheter, the outer electrode catheter comprising:
a catheter shaft having at least one electrode located at a distal end; and
a lumen which can receive the inner electrode catheter therein, the outer electrode catheter is adjustable or movable relative to the inner electrode catheter in an axial direction,
wherein the inner electrode catheter has a fixation element disposed at a distal end, the inner electrode catheter together with the fixation element forms an indifferent electrode whereby radio frequency catheter ablation occurs between the electrode and the indifferent electrode.

2. The electrode catheter device according to claim 1, wherein the fixation element of the inner electrode catheter is a helical screw.

3. (canceled)

4. The electrode catheter device according to claim 1, wherein the outer electrode catheter includes a red light emitting diode (LED) embedded proximate a distal end.

5. The electrode catheter device according to claim 1, wherein the outer electrode catheter carries at least one electrode for energy absorption.

6. The electrode catheter device according to claim 1, wherein the electrode of the outer electrode catheter is made of metal.

7. The electrode catheter device according to claim 2, wherein the electrode of the outer electrode catheter is designed in cylindrical shape or.

8. The electrode catheter device according to claim 2, wherein the inner electrode catheter comprises an electrically conducting helix shaped electrode such that the helix shaped electrode together with the helical screw from the indifferent electrode.

9. The electrode catheter device according to claim 1, wherein the shaft of the outer electrode catheter is made of flexible plastic.

10. The electrode catheter device according to claim 1, wherein high frequency voltage is applied between the electrode of the outer electrode cathether and the indifferent electrode thereby heating the subjacent tissue and creating an ablation path.

11. The electrode catheter device according to claim 1, wherein the fixation element of the inner electrode catheter is a magnet.

12. The electrode catheter device according to claim 1, wherein the outer electrode catheter includes a temperature sensor embedded proximate a distal end.

13. The electrode catheter device according to claim 1, wherein the outer electrode catheter carries at least one electrode for energy release.

14. The electrode catheter device according to claim 1, wherein the electrode of the outer electrode catheter is made of conductive plastic.

15. The electrode catheter device according to claim 1, wherein the electrode of the outer electrode catheter is designed in the shape of an electrically conducting segment.

16. A method for treatment of creating an ablation path, the method comprising:
guiding an inner electrode catheter to a desired target in the left atrium of a patient, wherein the inner electrode catheter has an electrically conducting helix;
fixing a distal end of the inner electrode catheter at an outer wall of the left atrium with a helical screw such that the electrically conducting helix of the inner electrode catheter and the helical screw from an indifferent electrode;
guiding an outer electrode catheter over the inner electrode catheter such that the outer electrode catheter concentrically encases the inner electrode catheter, wherein a shaft of the outer electrode catheter includes an electrode; and
applying a high frequency voltage between the electrode of the outer electrode catheter and the indifferent electrode thereby heating subjacent tissue and creating an ablation path.
